(19) United States
(12) Patent Application Publication
(10) Pub. No.: US 2026/0283574 A1
(43) Pub. Date: Sep. 24, 2026

(54) METHOD AND SYSTEM FOR PREDICTING COGNITIVE DECLINE AND FUNCTIONAL DISABILITY OF ALZHEIMER'S DISEASE USING WHOLE BRAIN WHITE MATTER PATTERN AND DETERMINING TYPE OF COGNITIVE IMPAIRMENT

(71) Applicants: DAEGU GYEONGBUK INSTITUTE OF SCIENCE AND TECHNOLOGY, Daegu (KR); AJOU UNIVERSITY INDUSTRY-ACADEMIC COOPERATION FOUNDATION, Suwon-si (KR)

(72) Inventors: DONGHA LEE, Daegu (KR); CHANG HYUNG HONG, Suwon-si (KR); SANG JOON SON, Suwon-si (KR); HYUN WOONG ROH, Suwon-si (KR)

(73) Assignees: DAEGU GYEONGBUK INSTITUTE OF SCIENCE AND TECHNOLOGY, Daegu (KR); AJOU UNIVERSITY INDUSTRY-ACADEMIC COOPERATION FOUNDATION, Suwon-si (KR)

(21) Appl. No.: 19/570,142

(22) Filed: Mar. 17, 2026

(30) Foreign Application Priority Data
Mar. 18, 2025 (KR) 10-2025-0034530

(51) Int. Cl. A61B 6/50; A61B 6/00; A61B 6/03; G01R 33/48; G16H 50/20

(52) U.S. Cl. CPC A61B 6/501; A61B 6/037; A61B 6/5217; A61B 6/5247; G01R 33/4806; G01R 33/481; G16H 50/20

(57) ABSTRACT

A method includes receiving, by input unit, a functional magnetic resonance imaging (fMRI) image and an amyloid positron emission tomography (amyloid-PET) image of a brain of a target person; extracting, by a preprocessing unit, multiple white matter masks from the fMRI image and the amyloid-PET image and generating first, second, and third matching images by using the multiple white matter masks; extracting, by an extraction unit, feature points respectively corresponding to a preset number of white matter regions by applying pre-constructed first, second, and third extraction models to the first matching image to the third matching image and extracting first white matter pattern to a third white matter pattern; and predicting, by a determination unit, the cognitive decline and the functional disability of the Alzheimer's disease and determining the type of the cognitive impairment.

### CROSS-REFERENCE TO RELATED APPLICATION

This application is based on and claims priority under 35 U.S.C. § 119 to Korean Patent Application No. 10-2025-0034530, filed on Mar. 18, 2025, in the Korean Intellectual Property Office, the disclosure of which is incorporated by reference herein in its entirety.

### BACKGROUND

The present disclosure relates to a method and system for predicting cognitive decline and functional disability of Alzheimer's disease using a whole brain white matter pattern and determining the type of cognitive impairment, and more specifically, to a method and system for extracting a brain white matter pattern by fusing a magnetic resonance image obtained by imaging the brain of a target person with a positron emission tomography (PET) image, predicting cognitive decline and functional disability related to Alzheimer's disease based on the extracted white matter pattern, and determining the type of cognitive impairment of the target person.

Cognitive impairment may be divided into subjective cognitive decline (SCD), mild cognitive impairment (MCI), vascular dementia (VaD), and Alzheimer's dementia (AD) depending on states of memory, attention, verbal ability, visuospatial ability, and judgment.

A cognitive impairment screening test (CIST), which is performed to determine cognitive impairment of a target person, has limitations, such as low reliability in accurately determining the cognitive impairment, and a diagnostic test (a neurocognitive test or a specialist's consultation) has limitations in that the test takes about two hours to perform and is relatively expensive, making the test difficult for the elderly to undergo.

In addition, by using amyloid positron emission tomography (amyloid-PET) images, after a substance that binds well to beta-amyloid is injected into a patient, the patient may be determined as either normal, mild cognitive impairment (MCI) which is likely to be dementia, or Alzheimer's disease based on changes in amyloid beta deposited in the brain.

Therefore, there is a need for technology that fuses together a magnetic resonance imaging (MRI) image and a positron emission tomography (PET) image of the brain of a target person to extract white matter patterns, and predicts whether the target person has Alzheimer's-related cognitive decline and functional disability based on the fused image, and determines the type of cognitive impairment.

The technology underlying the present disclosure is described in Korean Patent Publication No. 10-2024-0136778 (published on Sep. 19, 2024).

### SUMMARY

The present disclosure provides a method and system for extracting a brain white matter pattern by fusing a magnetic resonance image obtained by imaging the brain of a target person with a positron emission tomography (PET) image, predicting cognitive decline and functional disability related to Alzheimer's disease based on the extracted white matter pattern, and determining the type of cognitive impairment of the target person.

Additional aspects will be set forth in part in the description which follows and, in part, will be apparent from the description, or may be learned by practice of the presented embodiments of the disclosure.

According to an aspect of the present disclosure, a method of predicting cognitive decline and functional disability of Alzheimer's disease using a whole brain white matter pattern and determining a type of cognitive impairment includes receiving, by input unit, a functional magnetic resonance imaging (fMRI) image and an amyloid positron emission tomography (amyloid-PET) image of a brain of a target person; extracting, by a preprocessing unit, multiple white matter masks from the fMRI image and the amyloid-PET image and generating first, second, and third matching images by using the multiple white matter masks; extracting, by an extraction unit, feature points respectively corresponding to a preset number of white matter regions by applying pre-constructed first, second, and third extraction models to the first matching image to the third matching image and extracting first white matter pattern to a third white matter pattern based on the extracted feature points; and predicting, by a determination unit, the cognitive decline and the functional disability of the Alzheimer's disease and determining the type of the cognitive impairment by analyzing variability of white matter patterns between the first, second, and third white matter patterns of the target person and 1′, 2′, and 3′ white matter patterns of each of the cognitive impairment groups. The cognitive impairment groups include two or more of a subjective cognitive decline group, a mild cognitive impairment group, a vascular dementia group, and an Alzheimer's dementia group.

In the determining of the type of cognitive impairment, different multiple patient combinations may be selected for each of the cognitive impairment groups, variabilities 1′ white matter pattern to 3′ white matter pattern for each patient within a patient combination may be calculated, and variability of an average white matter pattern in a corresponding cognitive impairment group may be calculated, variabilities of white matter patterns between the first, second, and third white matter patterns of the target person and the 1′ white matter pattern to the 3′ white matter pattern of each of the cognitive impairment groups may be calculated, and the target person may be determined to belong to a cognitive impairment group having the variability of the average white matter pattern that differs the least from the calculated variabilities of the white matter patterns.

In the determining of the type of cognitive impairment, pairwise dissimilarity between the first, second, and third white matter patterns of the target person and the 1′ white matter pattern to the 3′ white matter pattern of each the cognitive impairment groups may be calculated by using an equation below, and the variabilities of white matter patterns are calculated based on the pairwise dissimilarity;

$$d ( x , y ) = \sqrt{{\sum }_{i}^{360} {( {x}_{i} - {y}_{i} )}^{2}}$$

wherein, d(x, y) is a correlation distance of the pairwise dissimilarity, x={x_{i=1, . . . , 360}|xᵢ∈ROIs} is average intensity of each of feature points of the first, second, and third white matter patterns of the target person, and y={y_{i=1, . . . , 360}|yᵢ∈ROIs} is average intensity of each of feature points of the 1′ white matter pattern to the 3′ white matter pattern of each of the cognitive impairment groups.

The generating of the first matching image to the third matching image may include: generating an image segmentation by extracting a T1-weighted image from the functional magnetic resonance image and separating a gray matter, a white matter, and a cerebrospinal fluid from the T1-weighted image; generating a white matter mask by separating the white matter from the image segmentation for the white matter; generating the first matching image by extracting a T2 FLAIR image from the magnetic resonance image and combining the T2 FLAIR image with the white matter mask; generating the second matching image by combining the received amyloid positron emission tomography image with the generated white matter mask; generating an ensemble image by applying a rigid body transformation to the amyloid positron emission tomography image and combining the amyloid positron emission tomography image with the T2 flare image; and generating a third matching image by combining the ensemble image with the generated white matter mask.

The extracting of the first, second, and third white matter patterns may include: extracting first feature points respectively corresponding to a preset number of white matter regions by applying the pre-constructed first extraction model to the first matching image; extracting the first white matter pattern by applying the first feature points to a preset analysis method; extracting second feature points respectively corresponding to a preset number of white matter regions by applying the pre-constructed second extraction model to the second matching image; extracting the second white matter pattern by applying the second feature points to the preset analysis method; extracting third feature points respectively corresponding to a preset number of white matter regions by applying the pre-constructed third extraction model to the third matching image; and extracting the third white matter pattern by applying the third feature points to the preset analysis method.

According to another aspect of the present disclosure, A system for predicting cognitive decline and functional disability of Alzheimer's disease using a whole brain white matter pattern and determining a type of cognitive impairment includes an input unit receives a functional magnetic resonance imaging (fMRI) image and an amyloid positron emission tomography (amyloid-PET) image of a brain of a target person; a preprocessing unit extracts multiple white matter masks from the fMRI image and the amyloid-PET image and generates first, second, and third matching images by using the multiple white matter masks; an extraction unit extracts feature points respectively corresponding to a preset number of white matter regions by applying pre-constructed first, second, and third extraction models to the first, second, and third matching images and extracting first, second, and third white matter patterns based on the extracted feature points; and a determination unit predicts the cognitive decline and the functional disability of the Alzheimer's disease and determines the type of the cognitive impairment by analyzing variability of white matter patterns between the first, second, and third white matter patterns of the target person and 1′, 2′, and 3′ white matter patterns of each of the cognitive impairment groups. The cognitive impairment groups include two or more of a subjective cognitive decline group, a mild cognitive impairment group, a vascular dementia group, and an Alzheimer's dementia group.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a configuration diagram of a system for predicting cognitive decline and functional disability of Alzheimer's disease and determining the type of the cognitive impairment of the Alzheimer's disease using a whole brain white matter pattern according to an embodiment of the present disclosure;

FIG. 2 is a flowchart of a method of predicting cognitive decline and functional disability of Alzheimer's disease using a whole brain white matter pattern and determining the type of the cognitive impairment according to another embodiment of the present disclosure;

FIG. 3 is a schematic diagram schematically illustrating a method of predicting cognitive decline and functional disability of Alzheimer's disease using a whole brain white matter patterns and determining the type of cognitive impairment according to another embodiment of the present disclosure;

FIG. 4 is a table showing clinical diagnosis results of participants according to another embodiment of the present disclosure;

FIGS. 5A and 5B are graphs illustrating accuracy of predicting cognitive decline and functional disability according to another embodiment of the present disclosure;

FIGS. 6A and 6B illustrates differences in variability of white matter patterns between cognitive impairment groups according to another embodiment of the present disclosure;

FIGS. 7A and 7B are graphs illustrating correlations between the calculated variabilities of white matter patterns and blood-based biomarkers according to another embodiment of the present disclosure;

FIG. 8 is a table showing an analysis result of a correlation between the calculated variability of a white matter pattern and a biomarker of a participant and clinical characteristics, according to another embodiment of the present disclosure; and

FIGS. 9A and 9B are graphs illustrating correlations between calculated variabilities of white matter patterns and cognitive scores of participants, according to another embodiment of the present disclosure.

### DETAILED DESCRIPTION

Hereinafter, preferred embodiments of the present disclosure will be described in detail with reference to the attached drawings. Herein, thicknesses of lines, sizes of components, and so on illustrated in the drawings may be exaggerated for the sake of clarity and convenience.

Also, the terms described below are defined based on functions of the present disclosure and may change depending on the intentions or practices of users or operators. Therefore, definitions of the terms should be based on the contents of the present disclosure.

In the following embodiments, it is described with specific examples that a system 100 for predicting cognitive decline and functional disability of Alzheimer's disease using a whole brain white matter pattern and determining the type of cognitive impairment is performed by a computing device including one or more memories or one or more processors which may perform the following processes.

Also, in the following embodiments, it is described with specific examples that cognitive impairment is a neurodegenerative brain disease and includes at least one of mild cognitive impairment (MCI), vascular dementia (VaD), and Alzheimer's dementia (AD). However, the present disclosure is not limited thereto.

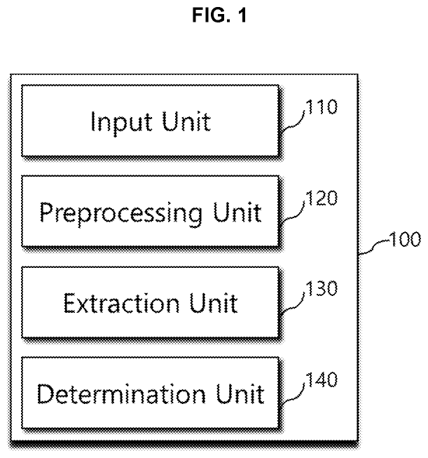

FIG. 1 is a configuration diagram illustrating the system 100 for predicting cognitive decline and functional disability of Alzheimer's disease using a whole brain white matter pattern and determining the type of cognitive impairment according to an embodiment of the present disclosure.

As illustrated in FIG. 1, the system 100 for predicting cognitive decline and functional disability of Alzheimer's disease using a whole brain white matter pattern and determining the type of cognitive impairment includes an input unit 110, a preprocessing unit 120, an extraction unit 130, and a determination unit 140.

First, the input unit 110 may receive a magnetic resonance image obtained by magnetic resonance imaging (MRI) or a positron emission tomography (PET) image. In this case, the magnetic resonance image may be a functional magnetic resonance image obtained by functional magnetic resonance imaging (fMRI), and the PET image may be an amyloid positron emission tomography (amyloid-PET) image.

Specifically, the input unit 110 may receive, from a user, a magnetic resonance image or a PET image of a part of the body including the head of a target person.

Next, the preprocessing unit 120 may extract multiple white matter masks from the received magnetic resonance image and the PET image and generate first, second, and third matching images by combining the extracted multiple white matter masks with the magnetic resonance image or the PET image.

Specifically, the preprocessing unit 120 may extract a T1-weighted image from the received magnetic resonance image and generate an image segmentation by separating a gray matter (GM), a white matter (WM), and a cerebrospinal fluid (CSF) from the T1-weighted image.

Also, the preprocessing unit 120 may generate a white matter mask (WM mask) by separating a WM from the image segmentation.

Also, the preprocessing unit 120 may extract a T2 fluid attenuated inversion recovery (FLAIR) image from the received magnetic resonance image and generate the first matching image by combining the T2 FLAIR image with the WM mask.

Also, the preprocessing unit 120 may generate the second matching image by combining the received amyloid-PET image with the WM mask.

Also, the preprocessing unit 120 may generate an ensemble image by matching the received amyloid-PET image to the T2 FLAIR image by using a rigid body transformation.

Also, the preprocessing unit 120 may generate the third matching image by combining the ensemble image with the WM mask.

Also, the preprocessing unit 120 may distinguish a preset number (for example, 360) of WM regions by applying a pre-constructed WM atlas to the generated WM mask. In this case, the WM atlas may be constructed by using a populational connection label map (pCLM) of the standardized entire white matter region and 360 regions of a human connectome project (HCP) brain map.

Next, the extraction unit 130 may extract feature points respectively corresponding to a preset number (for example, 360) of white matter regions by applying the pre-constructed first to third extraction models respectively to the generated first to third matching images and extract first to third white matter patterns by using the extracted feature points. In this case, the first to third extraction models may be constructed to extract feature points corresponding to the respective white matter regions from the first to third matching images by using the first to third matching images and the feature points corresponding to the respective white matter regions of the respective matching images as learning data.

Specifically, the extraction unit 130 may extract feature points respectively corresponding to the preset number (for example, 360) of white matter regions by applying the pre-constructed first extraction model to the first matching image.

Also, the extraction unit 130 may extract feature points respectively corresponding to the preset number (for example, 360) of white matter regions by applying the pre-constructed second extraction model to the second matching image.

Also, the extraction unit 130 may extract feature points respectively corresponding to the preset number (for example, 360) of white matter regions by applying a pre-constructed third extraction model to the third matching image.

Also, the extraction unit 130 may extract the first to third white matter patterns by applying the feature points extracted from the first to third matching images respectively to a preset analysis method (for example, support vector machine (SVM), logistic regression, decision tree, Naïve Bayes, k-nearest neighbor (KNN), learning vector quantization (LVQ), random forest, and so on).

Next, the determination unit 140 may analyze white matter pattern variability (white matter inter-subject variability: WM-ISV) between the extracted first to third white matter patterns of a target person and 1′ to 3′ white matter patterns of each cognitive impairment group (a subjective cognitive impairment group, an MCI group, a VaD group, and an AD group), predict cognitive decline and functional disability of Alzheimer's disease, and determine the type of cognitive impairment.

Specifically, the determination unit 140 may select multiple patient combinations that are different from each other for each cognitive impairment group, calculate the white matter pattern variability for each of the 1′ to 3′ white matter patterns between respective patients within the patient combinations, and then calculate an average white matter pattern variability of a corresponding cognitive impairment group. In this case, the determination unit 140 selects multiple patient combinations that are different from each other for all patients within the respective cognitive impairment groups.

Also, the determination unit 140 may calculate the white matter pattern variabilities between the first to third white matter patterns of a target person and white matter pattern variabilities between the calculated 1′ to 3′ white matter patterns of the respective cognitive impairment groups, and determine that the target person belongs to the cognitive impairment group with the an average white matter pattern variability that is the smallest difference from the calculated white matter pattern.

Also, the determination unit 140 may analyze the first to third white matter patterns of a target person to predict cognitive decline and functional disability when the target person has Alzheimer's disease.

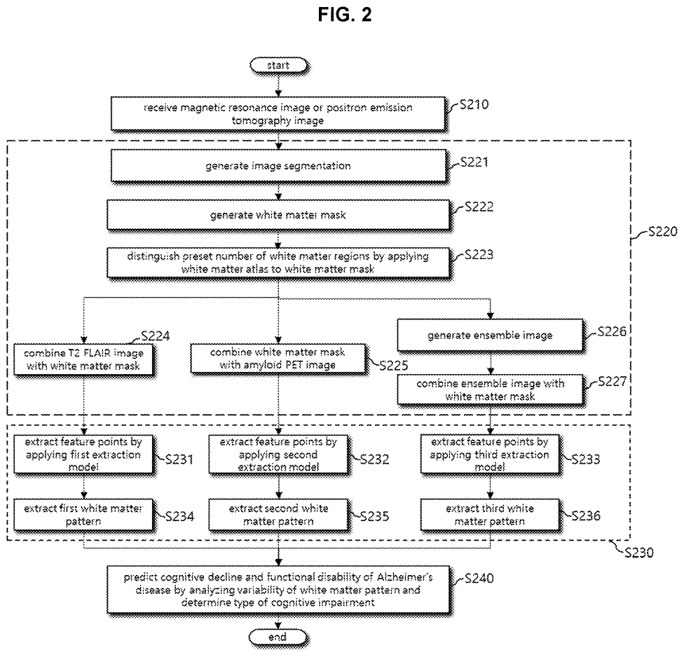

Hereinafter, a method of predicting cognitive decline and functional disability of Alzheimer's disease using the system 100 for predicting cognitive decline and functional disability using the whole brain white matter pattern and determining the type of cognitive impairment and determining the type of cognitive impairment, according to an embodiment of the present disclosure, will be described in more detail with reference to FIGS. 2 to 9B.

In the embodiment described below, a method for predicting cognitive decline and functional disability and determining the type of cognitive impairment of Alzheimer's disease using the whole brain white matter pattern is described as a specific example in which white matter patterns are extracted from 454 participants with at least one of subjective cognitive decline (or normal), mild cognitive impairment, vascular dementia, and Alzheimer's disease to determine the presence or absence of cognitive impairment. In this case, 454 participants underwent clinical interviews, neuropsychological tests, MRI, and PET.

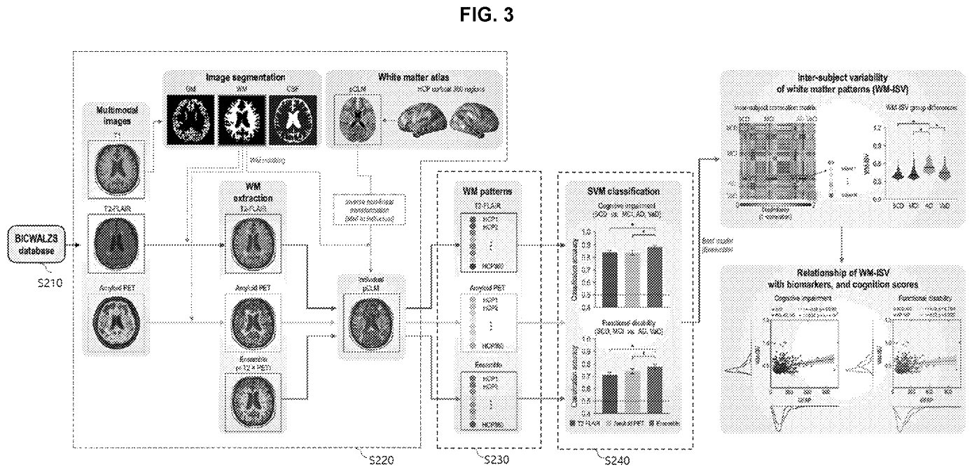

FIG. 2 is a flowchart illustrating the method of predicting cognitive decline and functional disability of Alzheimer's disease using a whole brain white matter pattern and determining the type of cognitive impairment according to another embodiment of the present disclosure, and FIG. 3 is a schematic diagram schematically illustrating the method of predicting cognitive decline and functional disability of Alzheimer's disease using a whole brain white matter pattern and determining the type of cognitive impairment according to another embodiment of the present disclosure.

As illustrated in FIGS. 2 and 3, the input unit 110 may receive a magnetic resonance image or a PET image (S210). In this case, the magnetic resonance image may be a functional magnetic resonance image, and the PET image may be an amyloid-PET image.

Specifically, the input unit 110 may receive, from the user, a magnetic resonance image or a PET image of a part of the body including the head of a target person.

Next, the preprocessing unit 120 may extract multiple white matter masks from the received magnetic resonance images and the received PET image and generate first, second, and third matching images by combining the extracted multiple white matter masks with the magnetic resonance image or the PET image (S220).

Specifically, the preprocessing unit 120 may extract a T1-weighted image from the received magnetic resonance image and generate an image segmentation by separating a GM, a WM, and a CSF from the T1-weighted image (S221).

Also, the preprocessing unit 120 may generate a WM mask by separating a WM from the image segmentation (S222).

Also, the preprocessing unit 120 may distinguish a preset number (for example, 360) of WM regions by applying a pre-constructed WM atlas to the generated WM mask (S223). In this case, the WM atlas may be constructed by using a pCLM of the standardized entire white matter region and 360 regions of an HCP brain map.

Also, the preprocessing unit 120 may generate the first matching image by extracting a T2 FLAIR image from the received magnetic resonance image and combining the T2 FLAIR image with the WM mask (S224).

Also, the preprocessing unit 120 may generate the second matching image by combining the received amyloid-PET image with the WM mask (S225).

Also, the preprocessing unit 120 may generate an ensemble image by matching the received amyloid-PET image to the T2 FLAIR image by using a rigid body transformation (S226).

Also, the preprocessing unit 120 may generate the third matching image by combining the ensemble image with the WM mask (S227).

Next, the extraction unit 130 may extract feature points respectively corresponding to a preset number (for example, 360) of white matter regions by applying the pre-constructed first to third extraction models respectively to the generated first to third matching images and extract first to third white matter patterns by using the extracted feature points (S230). In this case, the first to third extraction models may be constructed to extract feature points corresponding to the respective white matter regions from the first to third matching images by using the first to third matching images and the feature points corresponding to the respective white matter regions of the respective matching images as learning data.

Specifically, the extraction unit 130 may extract feature points respectively corresponding to the preset number (for example, 360) of white matter regions by applying the pre-constructed first extraction model to the first matching image (S231).

Also, the extraction unit 130 may extract feature points respectively corresponding to the preset number (for example, 360) of white matter regions by applying the pre-constructed second extraction model to the second matching image (S232).

Also, the extraction unit 130 may extract feature points respectively corresponding to the preset number (for example, 360) of white matter regions by applying a pre-constructed third extraction model to the third matching image (S233).

Also, the extraction unit 130 may extract the first to third white matter patterns by applying the feature points extracted from the first to third matching images respectively to preset analysis methods (for example, support vector machine (SVM), logistic regression, decision tree, Naïve Bayes, k-nearest neighbor (KNN), learning vector quantization (LVQ), random forest, and so on) (S234, S235, S236).

According to an embodiment of the present disclosure, the extraction unit 130 may average intensities of the 360 feature points extracted through the first to third extraction models by using the extracted first to third white matter patterns ({Yₛ}ₛ₌₁, . . . , N). In this case, average intensity (x={x_{i=1, . . . , 360}|xᵢ∈ROIs}) of the respective feature points may be calculated from a region of interest (ROI) of the HCP.

Next, the determination unit 140 may analyze white matter pattern variability (white matter inter-subject variability: WM-ISV) between the extracted first to third white matter patterns of a target person and the 1′ to 3′ white matter patterns of each cognitive impairment group (a subjective cognitive impairment group, an MCI group, a VaD group, and an AD group), predict cognitive decline and functional disability of Alzheimer's disease, and determine the type of cognitive impairment (S240).

Specifically, the determination unit 140 may select multiple patient combinations that are different from each other for each cognitive impairment group, calculate the white matter pattern variability for each of the 1′ to 3′ white matter patterns between respective patients within the patient combinations, and then calculate an average white matter pattern variability of a corresponding cognitive impairment group

In this case, the determination unit 140 may calculate a correlation distance d of pairwise dissimilarity for the 1′ to 3′ white matter patterns of each patient in a patient combination for each cognitive impairment group through Equation 1 below, and calculate variability of the white matter pattern based on the Equation 1.

$$d ( x , y ) = \sqrt{{\sum }_{i}^{360} {( {x}_{i} - {y}_{i} )}^{2}} \qquad Equation 1$$

Here, x={x_{i=1, . . . , 360}|xᵢ∈ROIs} is average intensity of each feature point of one patient in the patient combination, and y={y_{i=1, . . . , 360}|yᵢ∈ROIs} is average intensity of each feature point of another patient in the patient combination.

Also, the determination unit 140 may calculate the white matter pattern variabilities between the first to third white matter patterns of a target person and white matter pattern variabilities between the calculated 1′ to 3′ white matter patterns of the respective cognitive impairment groups, and determine that the target person belongs to the cognitive impairment group with the an average white matter pattern variability that is the smallest difference from the calculated white matter pattern.

In this case, the determination unit 140 may calculate the correlation distance d of pairwise dissimilarity for each cognitive impairment group by using Equation 1 above for the extracted first to third white matter patterns of a target person and the 1′ to 3′ white matter patterns of each cognitive impairment group (a subjective cognitive impairment group, an MCI group, a VaD group, and an AD group), and calculate variability of the white matter pattern based on the calculated correlation distance d.

Here, in Equation 1, x={x_{i=1, . . . , 360}|xᵢ∈ROIs} is average intensity of feature points of each of the first to third white matter patterns of the target person, and y={y_{i=1, . . . , 360}|yᵢ∈ROIs} is average intensity of feature points of each of the 1′ to 3′ white matter patterns of each cognitive impairment group.

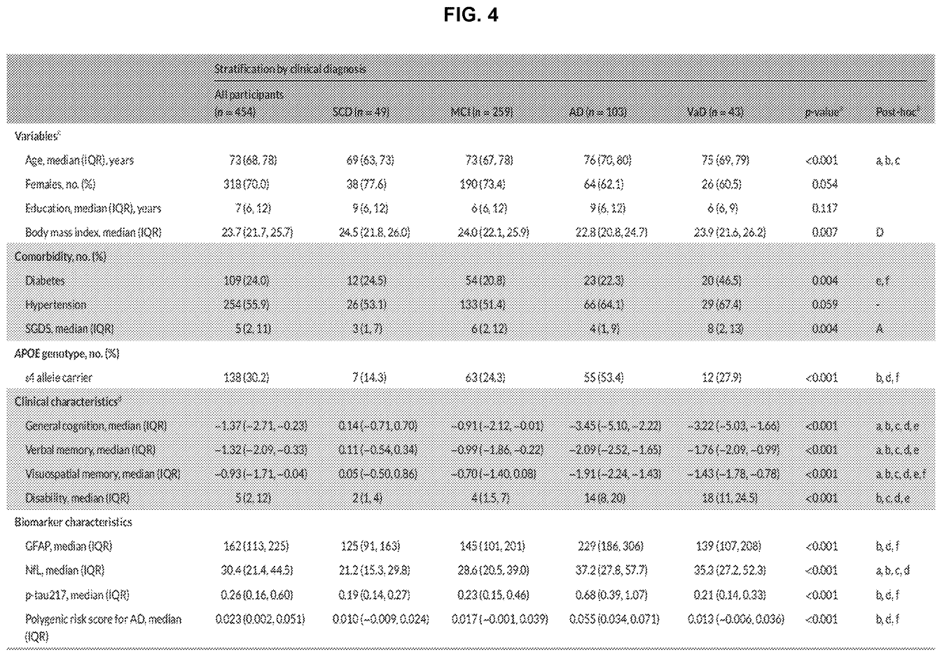

FIG. 4 is a table showing clinical diagnosis results of participants according to another embodiment of the present disclosure.

Referring to FIG. 4, participants in the AD group have a higher prevalence of ε4 allele carriers of apolipoprotein E (APOE) than participants in other groups, and participants in the VaD group have a higher incidence of diabetes than the participants in the AD group and the MCI group, and in cognition and impairment measurement results, including general cognitive ability, verbal and visuospatial memory ability, and impairment scores, a decreasing pattern is shown in the order of the subjective cognitive decline group, the MCI group, the AD group, and the VaD group.

Referring to FIG. 4, a blood-based neuronal damage biomarker (a neurofilament light chain (NfL)) gradually increases from the subjective cognitive decline group to the MCI group, the AD group, and the VaD group, and a blood-based inflammatory biomarker (glial-fiberillary-acidic-protein (GFAP)) and tau biomarker (pTau217) show the highest levels in the AD group.

Also, the determination unit 140 may analyze the first to third white matter patterns of a target person and predict cognitive decline and functional disability when the target person has Alzheimer's disease.

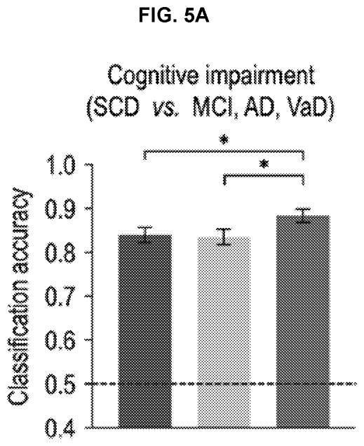

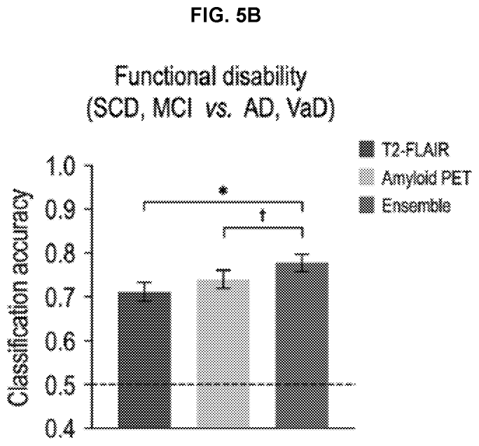

FIGS. 5A and 5B are graphs illustrating accuracy of predicting cognitive decline and functional disability according to another embodiment of the present disclosure.

As illustrated in FIG. 5A, it may be seen that the accuracy of predicting cognitive decline in Alzheimer's disease determined by the determination unit 140 by comparing the third white matter pattern and the 3′ white matter pattern is significantly higher than the accuracy determined by comparing the first white matter pattern with the 1′ white matter pattern or the second white matter pattern with the 2′ white matter pattern.

As illustrated in FIG. 5B, it may be seen that the accuracy of predicting functional disability of Alzheimer's disease determined by the determination unit 140 by comparing the third white matter pattern with the 3′ white matter pattern is significantly higher than the accuracy determined by comparing the first white matter pattern with the 1′ white matter pattern or the second white matter pattern with the 2′ white matter pattern.

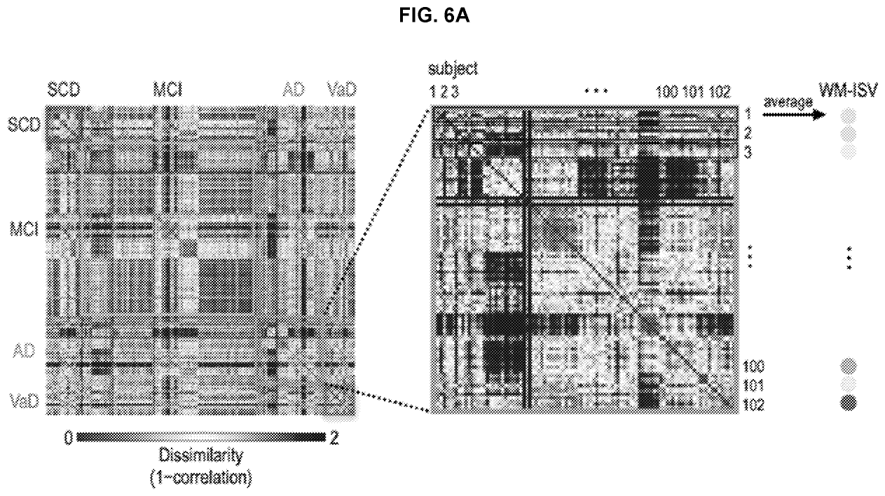

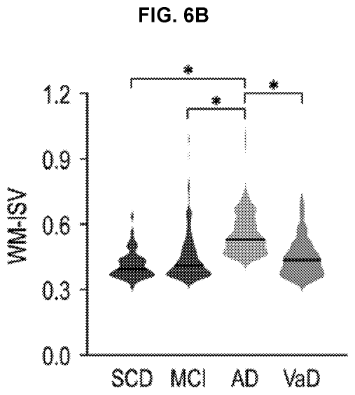

FIGS. 6A and 6B illustrates differences in variability of white matter patterns between cognitive impairment groups according to another embodiment of the present disclosure.

As illustrated in FIG. 6A, the determination unit 140 may calculate variability of a white matter pattern through a correlation distance of pairwise dissimilarity between respective cognitive impairment groups (a subjective cognitive impairment group, an MCI group, a VaD group, and an AD group) calculated by comparing the 3′ white matter patterns between respective patients within a patient combination for each cognitive impairment group.

As shown in FIG. 6B, the AD group shows significantly higher variability of a white matter pattern compared to other cognitive impairment groups.

Referring to FIGS. 6A and 6B, based on the correlation distance of pairwise dissimilarity, white matter patterns of the subjective cognitive decline group and the AD group show significant differences, and white matter patterns of the MCI group and the AD group also show significant differences, and accordingly, a white matter pattern is unique for each disease.

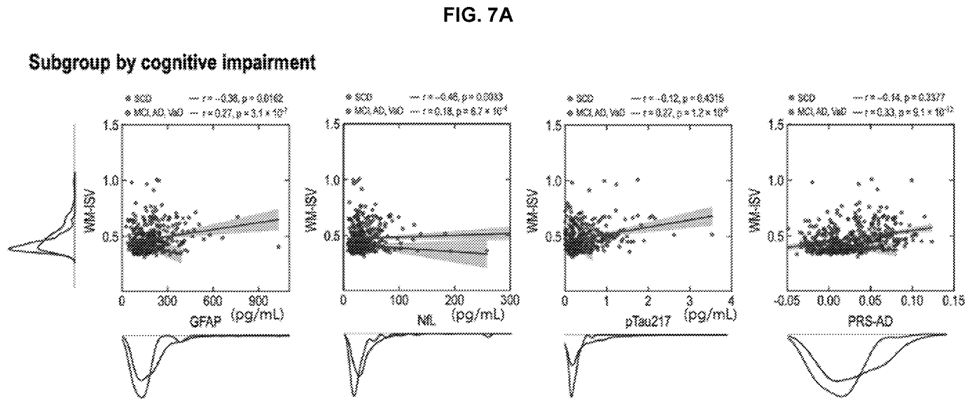

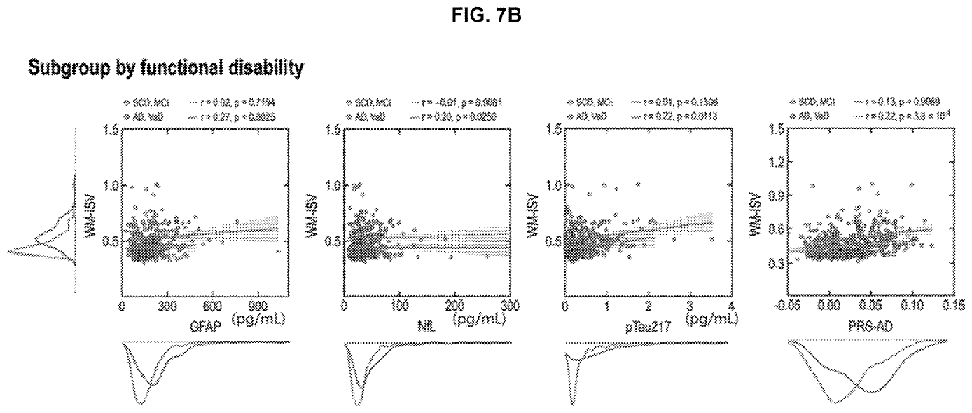

FIGS. 7A and 7B are graphs illustrating correlations between the calculated variabilities of white matter patterns and blood-based biomarkers according to another embodiment of the present disclosure.

Referring to FIG. 7A, in the correlations between the variabilities of white matter patterns according to cognitive impairment and blood-based biomarkers, the subjective cognitive decline group shows a negative correlation between a blood-based inflammatory biomarker, a blood-based neuronal damage biomarker, a tau biomarker, or a Polygenic Risk Score-Alzheimer's Dementia (PRS-AD) and variability of a white matter pattern, but the cognitive impairment group (the MCI group, the VaD group, and the AD group) shows a positive correlation.

Referring to FIG. 7B, in the correlations between the variabilities of white matter patterns according to functional disability and blood-based biomarkers, the AD group and the VaD group show a positive correlation between blood-based inflammatory biomarkers or blood-based neuronal damage biomarkers and the variabilities of white matter patterns, but the subjective cognitive decline and the MCI group do not show significant correlation.

Referring to FIG. 7B, the subjective cognitive decline group shows a negative correlation between a tau biomarker or PRS-AD and the variabilities of white matter patterns, but the cognitively impair groups (the MCI group, the VaD group, and AD group) show a positive correlation.

Accordingly, cognitive impairment of a target person and the type of cognitive impairment may be determined by using variability of white matter patterns because each group shows a correlation between a biomarker and variability of a white matter pattern.

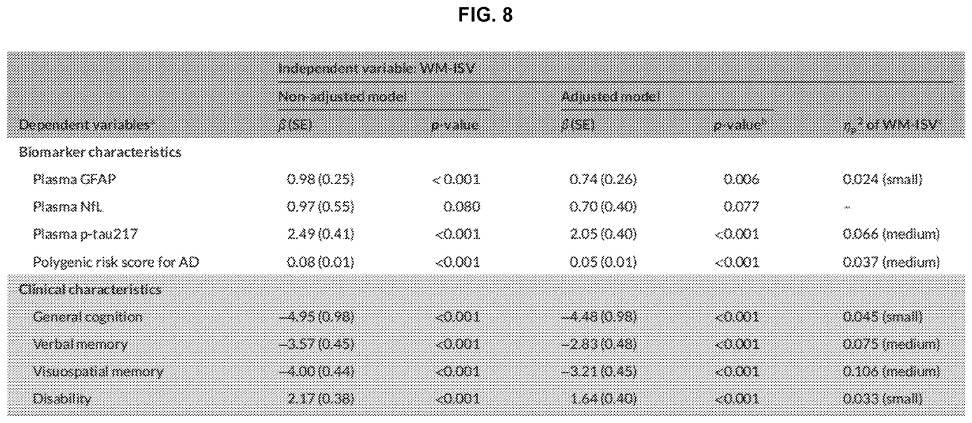

FIG. 8 is a table showing an analysis result of a correlation between the calculated variability of a white matter pattern and a biomarker of a participant and clinical characteristics, according to another embodiment of the present disclosure.

Referring to FIG. 8, there is a significant correlation between clinical characteristics (general cognitive ability, verbal and visuospatial memory ability, and impairment score) of each group and the variability of white matter patterns.

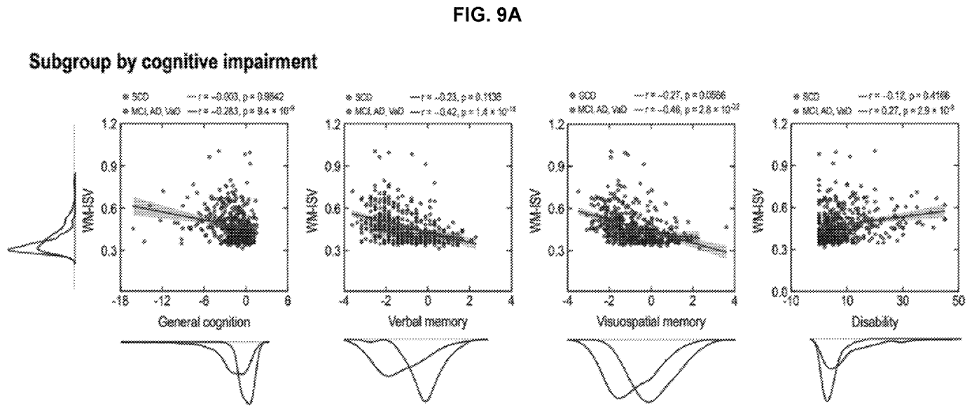

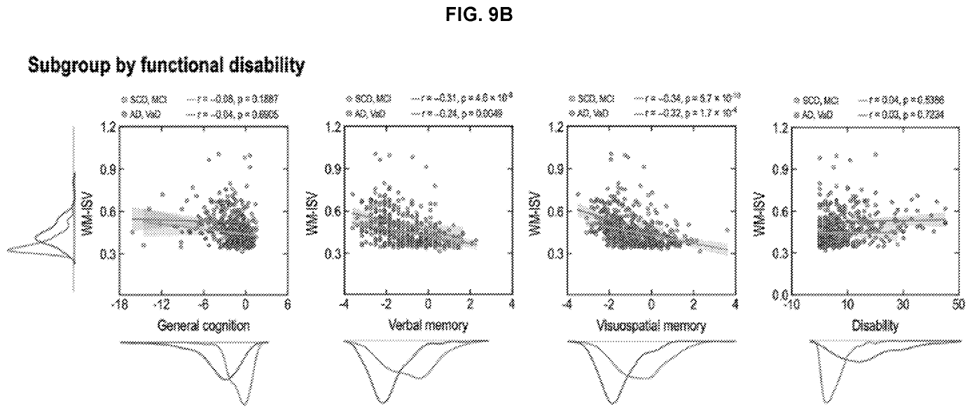

FIGS. 9A and 9B are graphs illustrating correlations between calculated variabilities of white matter patterns and cognitive scores of participants, according to another embodiment of the present disclosure.

Referring to FIGS. 8 and 9A, the variability of white matter patterns and general cognitive ability show a negative correlation in variability of white matter patterns and clinical characteristics according to cognitive impairment, and verbal and visuospatial memory ability and variability of white matter patterns show a negative correlation in a subjective cognitive decline group, and cognitive impairment groups (an MCI group, a VaD group, and an AD group) show a positive correlation.

Referring to FIGS. 8 and 9B, general cognitive ability and verbal and visuospatial memory ability show a negative correlation regardless of the presence or absence of functional disability in the variability of white matter patterns and clinical characteristics according to cognitive impairment, and impairment scores show a positive correlation regardless of functional disability.

That is, as illustrated in FIG. 8, by utilizing a generalized linear model and adjusting covariance, the variability of white matter patterns shows a significant correlation with general cognitive ability, verbal and visuospatial memory ability and the impairment scores.

According to the embodiments of the present disclosure described above, white matter patterns extracted from images, in which structural brain MRI images, amyloid-PET (AET) images, and structural MRI and AET images are combined together, may be used as multimodal brain imaging biomarkers.

Also, multimodal brain imaging biomarkers may be used to determine chronic cerebrovascular diseases (Alzheimer's disease, vascular dementia, and so on).

While the present disclosure is described with reference to the embodiments illustrated in the drawings, the embodiments are only examples, and those skilled in the art will understand that various modifications and equivalent embodiments may be derived therefrom. Therefore, the true technical protection scope of the present disclosure should be determined by the technical idea of the following claims.

What is claimed is:

1. A method of predicting cognitive decline and functional disability of Alzheimer's disease using a whole brain white matter pattern and determining a type of cognitive impairment, the method comprising:
receiving, by input unit, a functional magnetic resonance imaging (fMRI) image and an amyloid positron emission tomography (amyloid-PET) image of a brain of a target person;
extracting, by a preprocessing unit, multiple white matter masks from the fMRI image and the amyloid-PET image and generating first, second, and third matching images by using the multiple white matter masks;
extracting, by an extraction unit, feature points respectively corresponding to a preset number of white matter regions by applying pre-constructed first, second, and third extraction models to the first matching image to the third matching image and extracting first white matter pattern to a third white matter pattern based on the extracted feature points; and
predicting, by a determination unit, the cognitive decline and the functional disability of the Alzheimer's disease and determining the type of the cognitive impairment by analyzing variability of white matter patterns between the first, second, and third white matter patterns of the target person and 1′, 2′, and 3′ white matter patterns of each of the cognitive impairment groups,
wherein the cognitive impairment groups include two or more of a subjective cognitive decline group, a mild cognitive impairment group, a vascular dementia group, and an Alzheimer's dementia group.

2. The method of claim 1, wherein,
in the determining of the type of cognitive impairment,
different multiple patient combinations are selected for each of the cognitive impairment groups, variabilities 1′ white matter pattern to 3′ white matter pattern for each patient within a patient combination are calculated, and variability of an average white matter pattern in a corresponding cognitive impairment group is calculated, variabilities of white matter patterns between the first, second, and third white matter patterns of the target person and the 1′ white matter pattern to the 3′ white matter pattern of each of the cognitive impairment groups are calculated, and
the target person is determined to belong to a cognitive impairment group having the variability of the average white matter pattern that differs the least from the calculated variabilities of the white matter patterns.

3. The method of claim 2, wherein,
in the determining of the type of cognitive impairment, pairwise dissimilarity between the first, second, and third white matter patterns of the target person and the 1′ white matter pattern to the 3′ white matter pattern of each the cognitive impairment groups is calculated by using an equation below, and the variabilities of white matter patterns are calculated based on the pairwise dissimilarity;
$$d ( x , y ) = \sqrt{{\sum }_{i}^{360} {( {x}_{i} - {y}_{i} )}^{2}}$$
wherein, d(x, y) is a correlation distance of the pairwise dissimilarity, x={x_{i=1, . . . , 360}|xᵢ∈ROIs} is average intensity of each of feature points of the first, second, and third white matter patterns of the target person, and y={y_{i=1, . . . , 360}|yᵢ∈ROIs} is average intensity of each of feature points of the 1′ white matter pattern to the 3′ white matter pattern of each of the cognitive impairment groups.

4. The method of claim 1, wherein the generating of the first matching image to the third matching image includes:
generating an image segmentation by extracting a T1-weighted image from the functional magnetic resonance image and separating a gray matter, a white matter, and a cerebrospinal fluid from the T1-weighted image;
generating a white matter mask by separating the white matter from the image segmentation for the white matter;
generating the first matching image by extracting a T2 FLAIR image from the magnetic resonance image and combining the T2 FLAIR image with the WM mask;
generating the second matching image by combining the received amyloid positron emission tomography image with the generated white matter mask;
generating an ensemble image by applying a rigid body transformation to the amyloid positron emission tomography image and combining the amyloid positron emission tomography image with the T2 flare image; and
generating a third matching image by combining the ensemble image with the generated white matter mask.

5. The method of claim 1, wherein the extracting of the first, second, and third white matter patterns includes:
extracting first feature points respectively corresponding to a preset number of white matter regions by applying the pre-constructed first extraction model to the first matching image;
extracting the first white matter pattern by applying the first feature points to a preset analysis method;
extracting second feature points respectively corresponding to a preset number of white matter regions by applying the pre-constructed second extraction model to the second matching image;
extracting the second white matter pattern by applying the second feature points to the preset analysis method;
extracting third feature points respectively corresponding to a preset number of white matter regions by applying the pre-constructed third extraction model to the third matching image; and
extracting the third white matter pattern by applying the third feature points to the preset analysis method.

6. A system for predicting cognitive decline and functional disability of Alzheimer's disease using a whole brain white matter pattern and determining a type of cognitive impairment, the system comprising:
an input unit receives a functional magnetic resonance imaging (fMRI) image and an amyloid positron emission tomography (amyloid-PET) image of a brain of a target person;
a preprocessing unit extracts multiple white matter masks from the fMRI image and the amyloid-PET image and generates first, second, and third matching images by using the multiple white matter masks;
an extraction unit extracts feature points respectively corresponding to a preset number of white matter regions by applying pre-constructed first, second, and third extraction models to the first, second, and third matching images and extracting first, second, and third white matter patterns based on the extracted feature points; and
a determination unit predicts the cognitive decline and the functional disability of the Alzheimer's disease and determines the type of the cognitive impairment by analyzing variability of white matter patterns between the first, second, and third white matter patterns of the target person and 1′, 2′, and 3′ white matter patterns of each of the cognitive impairment groups,
wherein the cognitive impairment groups include two or more of a subjective cognitive decline group, a mild cognitive impairment group, a vascular dementia group, and an Alzheimer's dementia group.

7. The system of claim 6, wherein
the determination unit selects different multiple patient combinations for each of the cognitive impairment groups, calculates variabilities 1′ white matter pattern to 3′ white matter pattern for each patient within a patient combination, calculates variability of an average white matter pattern in a corresponding cognitive impairment group, calculates variabilities of white matter patterns between the first, second, and third white matter patterns of the target person and the 1′ white matter pattern to the 3′ white matter pattern of each of the cognitive impairment groups, and determines the target person as belonging to a cognitive impairment group having the variability of the average white matter pattern that differs the least from the calculated variabilities of the white matter patterns.

8. The system of claim 7, wherein
the determination unit calculates pairwise dissimilarity between the first, second, and third white matter patterns of the target person and the 1′ white matter pattern to the 3′ white matter pattern of each the cognitive impairment groups by using an equation below, and calculates the variabilities of white matter patterns based on the pairwise dissimilarity;
$$d ( x , y ) = \sqrt{{\sum }_{i}^{360} {( {x}_{i} - {y}_{i} )}^{2}}$$
wherein, d(x, y) is a correlation distance of the pairwise dissimilarity, x={x_{i=1, . . . , 360}|xᵢ∈ROIs} is average intensity of each of feature points of the first, second, and third white matter patterns of the target person, and
y={y_{i=1, . . . , 360}|yᵢ∈ROIs} is average intensity of each of feature points of the 1′ white matter pattern to the 3′ white matter pattern of each of the cognitive impairment groups.

9. The system of claim 6, wherein
the preprocessing unit generates an image segmentation by extracting a T1-weighted image from the functional magnetic resonance image and separating a gray matter, a white matter, and a cerebrospinal fluid from the T1-weighted image, generates a white matter mask by separating the white matter from the image segmentation for the white matter, generates the first matching image by extracting a T2 FLAIR image from the magnetic resonance image and combining the T2 FLAIR image with the WM mask, generates the second matching image by combining the received amyloid positron emission tomography image with the generated white matter mask, generates an ensemble image by applying a rigid body transformation to the amyloid positron emission tomography image and combining the amyloid positron emission tomography image with the T2 flare image, and generates a third registered image by combining the ensemble image with the generated white matter mask.

10. The system of claim 6, wherein
the extraction unit extracts first feature points respectively corresponding to a preset number of white matter regions by applying the pre-constructed first extraction model to the first matching image, extracts the first white matter pattern by applying the first feature points to a preset analysis method, extracts second feature points respectively corresponding to a preset number of white matter regions by applying the pre-constructed second extraction model to the second matching image, extracts the second white matter pattern by applying the second feature points to the preset analysis method, extracts third feature points respectively corresponding to a preset number of white matter regions by applying the pre-constructed third extraction model to the third matching image, and extracts the third white matter pattern by applying the third feature points to the preset analysis method.
